(19) United States
(12) Patent Application Publication
(10) Pub. No.: US 2007/0104732 A1
(43) Pub. Date: May 10, 2007

(54) Ligand-pseudoreceptor system for generation of adenoviral vectors with altered tropism

(76) Inventors: Bernard Massie, Laval (CA); Yue Zeng, LaSalle (CA); Maureen O'Connnor-McCourt, Beaconsfield (CA)

Correspondence Address: OGILVY RENAULT LLP, 1981 MCGILL COLLEGE AVENUE, SUITE 1600, MONTREAL, QC, H3A2Y3, CA

(21) Appl. No.: 10/576,500

(22) PCT Filed: Oct. 22, 2004
(86) PCT No.: PCT/CA04/01794

§ 371 Date: Nov. 22, 2006

Related U.S. Application Data
(60) Provisional application No. 60/514,532, filed on Oct. 24, 2003.

(51) Int. Cl. A61K 39/12; C12Q 1/70; C12N 7/00; C12N 15/86

(52) U.S. Cl. 424/204.1; 435/5; 435/235.1; 435/456

(57) ABSTRACT

In accordance with the present invention, there is provided a modified virus ablated of its natural receptors interactions with an unmodified or non-naturally occurring cell, said modified virus comprising a non-native polypeptide, said modified virus having an altered tropism conferred by said non-native peptide, and replicating only in cells that can interact with said non-native peptide, said virus being incapable of infecting a cell through a CAR-dependent entry pathway. There is also provided a modified cell line for replicating the modified virus. These two together can be advantageously put into practice in the field of medicine and more particularly in gene therapy.

### TECHNICAL FIELD

The present invention relates to a new retargeted virus, to corresponding permissive propagation cells and to a new system for the propagation of native tropism-ablated adenoviral vectors by using a different ligand/pseudo-receptor pair, allowing retargeting of said vectors.

### BACKGROUND OF THE INVENTION

Adenoviruses (Ad) are able to infect a variety of cell types, but their wide tropism is a limitation for certain applications such as cancer therapy, because both the normal and diseased cells are transduced. The unspecific transduction has not only the negative effect on normal cell function, but also decreases the amount of therapeutic viruses delivered to the diseased cells. Therefore, targeted vectors have been developed in order to selectively localize gene expression to the tissue of interest.

Uptake of the Ad vectors (AdV) derived from serotypes 2 and 5 is a two-stage process, which involves an initial interaction of the viral fiber protein with cellular receptors such as CAR (coxsackievirus and Ad receptor) (Bergelson J M, et al., Science, 275(5304): 1320-1323,1997; and Defer C, B. M., et al., J Virol., 64(8): 3661-3673,1990). The CAR binding domain is localized on the knob region of fiber (Santis G, et al., J Gen Virol, 80: 1519-1527, 1999). Ad binding is then followed by the internalization of the virus, which is mediated by the interaction of the RGD motif of the penton base (viral protein) with secondary cellular receptors identified as αv integrins. This step allows virus internalization via receptor-mediated endocytosis (Wickham T J, et al., Cell, 73(2): 309-319, 1993). Based on the virus entry mechanisms, several strategies were developed to create new CAR-independent entry pathway for the re-targeting of AdVs.

Several studies were undertaken using either chimeric fibers or exchanging fibers from different serotypes as a simple way to alter AdV tropism (Shayakhmetov D M, et al., J Virol, 74(6): 2567-2583, 2000) since it was suggested that they might recognize different receptors and consequently have different tropism. However, true targeting of AdVs requires the ablation of the vector interaction with their natural receptors, as well as the redirection of the vector to another type of receptor, which is specific to the target cells.. Mutagenesis of the fiber has been done to ablate virus-CAR interaction in vitro. Substitutions within knob region of fiber dramatically reduce the transduction of various CAR-positive cell lines (Alemany R and C. D T., Gene Ther, 8(17): 1347-1353, 2001; Jakubczak J L, et al., J virol, 75(6): 2972-2981, 2001; Roelvink P W, et al., Science, 286(5444): 1568-1571, 1999; and Leissner P, et al., Gene Ther, 8(1): 49-57, 2001). The shaft domain of the fiber has also been changed to modify Ad natural tropism. It was shown that the high transduction efficiency of the liver and the spleen was dramatically reduced by the replacement of a shorter shaft within the fiber (Nakamura T, et al., J Virol, 77(4): 2512-2521, 2003). On the other hand, in order to redirect AdVs to target cells, viral capsid proteins (fiber, penton base, and hexon) were genetically modified by insertion of new ligands or chemically combined with ligands of specific receptors. Ligands such as poly-lysine, RGD motif, NGR peptides, epithelium growth factor (EGF) and gastrin releasing peptide (GRP), respectively targeting heparan sulfates, integrins, aminopeptidase N (CD13), EGF and GRP receptors, have been evaluated for their capacity to alter viral tropism (Wickham T J, et al., Cell, 73(2): 309-319, 1993; Krasnykh V, et al., J Virol, 72(3): 1844-1852, 1998; Vanderkwaak T J, et al., Gynecol Oncol, 74(2): 227-234, 1999; Dmitriev I, et al., J Virol, 74(15): 6875-6884, 2000; and Hong S S, et al., Virology, 262(1): 163-177, 1999). Recently, a synthetic 33-amino-acid immunoglobulin G (IgG)-binding domain derived from staphylococcal protein A was inserted into the Ad fiber making possible a directed gene transfer to a wide variety of cell types by simply changing the target-specific antibody (Volpers C, et al., J Virol, 77(3): 2093-2104, 2003).

As a result of the ablation of binding to its native receptors, AdV can no longer be produced in the current complementing cell lines; hence the need for new packaging cells. One approach is to construct cell lines expressing an alternate pseudoreceptor, which allows the binding and uptake of targeted vectors. Thus, in addition to the targeting ligand incorporated into the AdV capsid for cell-specific transduction, another pseudoreceptor-binding ligand should also be inserted in the vector for their entry and propagation in packaging cells. This pair of de novo designed pseudoreceptor-ligand would be completely artificial, such that no natural receptors could be used for entry of the vector through the new ligand in vivo. For example, a cell line expressing the pseudoreceptor made of a membrane-anchored single-chain antibody against hemagglutinin (HA) was shown to be able to support HA-tagged AdV production (Einfeld D A, et al., J Virol, 73(11): 9130-9136, 1999). Another cell line expressing the pseudoreceptor, which contains an anti-His sFv, allowed the infection of AdV carrying histidine-incorporated fiber (Douglas J T, et al., Nat Biotechnol, 17(5): 470-475,1999).

The overall strategy for the development of Ad vectors (AdV) for the delivery of transgenes in specific tissues relies both on the ablation of Ad native tropism and the introduction of new tropism for target cells. In the process, AdVs ablated for their natural receptor interactions would be unable to grow in current cell lines. Consequently such ablated AdVs require new packaging cells for their generation.

It would be highly desirable to be provided with a new modified virus ablated of its native tropism, which could be used as a “universal virus” that could be retargeted to specific targets.

### SUMMARY OF THE INVENTION

One aim of the present invention is to provide a new modified virus ablated of its native tropism, which could be used as a “universal virus” that could be retargeted to specific targets.

Another aim of the present invention is to provide a new modified virus ablated of its native tropism, which cannot replicate in most naturally-occurring cells.

It is thus an object of the invention to establish a new modified virus ablated of its native tropism and so modified as to be propagation or replication incompetent in most cells.

Another aim of the present invention is to provide new cells that have been modified to be infection permissive and to allow replication of the virus of the present invention.

In accordance with the present invention there is provided a modified virus ablated of its natural receptors interactions with an unmodified or non-naturally occurring cell, said modified virus comprising a non-native polypeptide, said modified virus having an altered tropism conferred by said non-native peptide, and replicating only in cells that can interact with said non-native peptide, said virus being incapable of infecting a cell through a CAR-dependent entry pathway.

The modified virus can be made from or derived from, for example a virus selected from the group consisting of adenovirus, retrovirus, lentivirus, adeno-associated virus, Reoviridae, Picornaviridae, Parvoviridae, Papovaviridae and Caliciviridae, more preferably from human adenovirus such as human adenovirus serotype 2 or 5.

In one embodiment of the invention, the non-native polypeptide replaces, is incorporated into, or forms a fusion protein with, a viral protein component (such as an adenoviral fiber protein) of the wild type virus.

In one embodiment of the invention, the non-native polypeptide is incorporated into an adenoviral fiber protein such that the wild-type fiber knob or cell binding domain thereof is removed.

In one embodiment of the invention, the non-native polypeptide is or comprises a combinatorial protein or an affibody.

In one embodiment of the invention, the non-native polypeptide comprises one or more sequence from a bacterial receptor ligand.

In one embodiment of the invention, the non-native polypeptide comprises at least one repeat of a sequence as set forth in SEQ ID NO:1.

In another embodiment of the invention, the non-native polypeptide comprises at least one repeat of a sequence as set forth in SEQ ID NO:2.

In one embodiment of the invention, the non-native polypeptide binds a non-naturally occurring production cell or permissive cell.

In one embodiment of the invention, the modified virus further comprises a retargeting adapter comprising i) a binding moiety for binding the non-native polypeptide and ii) a further binding moiety of a receptor for retargeting said virus on cells expressing said receptor.

In a further embodiment of the invention, the non-native polypeptide comprises at least one repeat of a sequence as set forth in SEQ ID NO:1 and said binding moiety for binding the non-native polypeptide comprises at least one repeat of SEQ ID NO:2.

In another embodiment of the invention, the non-native polypeptide comprises at least one repeat of a sequence as set forth in SEQ ID NO:2 and said binding moiety for binding the non-native polypeptide comprises at least one repeat of SEQ ID NO:1.

The adapter in one embodiment binds to the non-native polypeptide through non-covalent physical forces selected from the group consisting of van der waals forces, electrostatic forces, stacking interactions, hydrogen bonding and steric fit.

The non-native polypeptide may optionally comprise a cleavage site positioned in a location that enables a further binding moiety of a receptor to be added on the modified virus for retargeting said virus on cells expressing said receptor.

The binding moiety is preferably capable of binding to a cell specific ligand.

In one embodiment of the invention, the modified virus further comprises a site for insertion of one or more desired therapeutic genes or nucleic acid molecules.

In accordance with the present invention, there is provided a cell containing a modified virus as defined above.

Still in accordance with the present invention, there is provided a permissive cell for a modified virus as defined above, which is capable of being cultured to propagate said modified virus.

Further in accordance with the present invention, there is still provided a non-naturally occurring permissive cell expressing a surface receptor recognizing or binding a non-native polypeptide as defined above.

In accordance with the present invention, there is also provided a non-naturally occurring permissive cell expressing a surface receptor recognizing or binding a non-native polypeptide as defined above, wherein said surface receptor comprises at least one copy of the amino acid sequence as set forth in SEQ ID NO:2 or SEQ ID NO:1, as the case may be, depending on the other element of the binding pair.

In accordance with the present invention, there is also provided a method for producing a modified virus as defined above in cell culture, comprising the steps of: i) genetically modifying a virus to produce a modified virus ablated of its natural receptors interactions with an unmodified or non-naturally occurring cell, said modified virus comprising a non-native polypeptide, said modified virus having an altered tropism conferred by said non-native peptide, and replicating only in cells that can interact with said non-native peptide; ii) infecting permissive cells with said modified virus; and iii) culturing said cells to produce the virus. The method may further comprise a step of iv) harvesting the modified virus produced. The method may additionally comprise a step of v) purifying the modified virus produced.

The modified virus of the present invention can be use in therapy.

In accordance with the present invention there is also provided the use of the modified virus as defined above in the preparation of a medicament for the treatment of tumor cells or proliferating cells.

Still in accordance with the present invention, there is further provided a pharmaceutical composition comprising a modified virus as defined above and a pharmaceutically acceptable carrier or excipient.

There is also provided in accordance with the present invention a reagent kit comprising a modified virus and a cell, both as defined herein.

In accordance with the present invention, there is also provided a medicament or a precursor thereof comprising a virus as defined herein.

Still in accordance with the present invention, there is also provided the use of a virus as defined herein for the preparation of a medicament or a precursor thereof for treating or preventing genetic diseases, tumor diseases, autoimmune diseases or infectious diseases.

### BRIEF DESCRIPTION OF THE DRAWINGS

FIG. 1 is a schematic diagram of the EGFR-Ecoil pseudoreceptor;

FIGS. 2A and 2B illustrate the stable expression of the pseudoreceptor EGFR-Ecoil by flow cytometry analysis (FIG. 2A) and by Western blot analysis (FIG. 2B);

FIG. 3 illustrates the growth profile of 293E cells;

FIG. 4 illustrates an analysis of fiber-Kcoil transit-expression by western blot;

FIG. 5 illustrates the transduction of 293 and 293E cells by AdFK4m/GFP and Ad/GFP;

FIGS. 6A and 6B illustrates specific transduction of 293 and 293E cells by AdFK4m/GFP and Ad/GFP;

FIG. 7 illustrates the virus growth kinetics in cells 293E;

FIG. 8 illustrates immunoblot analysis showing the trimer form of modified-fiber;

FIGS. 9A and 9B illustrate the gene transfer profile of AdFK4m/GFP and AdK4mmRGD/GFP in 293 and 293E cells respectively; and

FIGS. 10A and 10B illustrate the gene transfer profile of AdFK4m/GFP and AdK4mmRGD/GFP in HeLa and A549 cells respectively.

### DETAILED DESCRIPTION OF THE PREFERRED EMBODIMENT

In accordance with the present invention, there is provided a new modified virus and its replicating cell. The idea behind the present invention for modifying the virus was to render the virus incompetent in duplicating in naturally-occurring cells, such as mammalian cells. Therefore the virus was modified to prevent binding to its natural receptors. However, without binding to its natural receptors on cells, the modified virus could not be reproduced or replicated. Thus, the virus was further modified to have a first artificial binding element of a binding pair, and a new cell was also constructed to have on its surface the other element of the binding pair. The binding pair was carefully chosen to have an appropriate affinity with each other to ensure efficient delivery of the viral vector.

Therefore, according to one aspect of the invention, two de novo-designed peptides (E-coil and K-coil), which interact with each other with high affinity were constructed to establish a new receptor-ligand system. These peptides each contains from 1 to 5 repeats of EVSALEK (SEQ ID NO: 1) and kVSALKE (SEQ ID NO:2) sequences, respectively. A pseudoreceptor, composed of E-coil fused with the transmembrane and cytoplasmic domains of EGFR was developed. 293 cells expressing such pseudoreceptor (293E) were shown to efficiently propagate a CAR-ablated AdV containing the complementary K-coil motif incorporated in. its fiber knob (AdFK4m). Furthermore, it has been shown herein that virus entry is mediated in a CAR-independent pathway via E-coil/K-coil interaction. Furthermore, the fiber of such modified virus could be further modified by the insertion of a ligand (RGD motif) for targeting to new tropism. These results demonstrate that the packaging cell line 293E and AdFK4m constitute a useful platform for the generation of re-targeted AdVs.

The new virus and its corresponding propagation cells constitute a useful tools in gene therapy and more particularly in cancer therapy. One skilled in the art will appreciate that a further ligand can be attached to the virus so as to retarget the virus to a specific cell that bears the receptor for this further ligand. This further ligand can be inserted in the virus through genetic manipulation for the virus to express this further ligand. Alternatively, the further ligand can be attached to a linker which would recognize the first element of the binding pair and bind thereto. In such an embodiment, the linker comprises for example the other element of the binding pair to which is attached the further ligand. Thus, the first element of the binding pair now binds in the presence of the linker to the other element of the binding pair exposing at the end of said linker the further ligand. In such an embodiment, the modified virus do not normally replicate in a natural environment, but requires the modified cells to replicate. Once replication is achieved to a desired level, a linker comprising the other element of the binding pair and the further ligand specifically chosen for a specific application is attached to the modified virus for targeting the virus to a specific type of cells determined by the further ligand chosen.

It will also be appreciated that specificity of the binding elements can be modified by either increasing the length of the sequence of the elements of by repeating in tandem the elements on the virus and at the surface of the modified cells or on the linker.

The present invention will be more readily understood by referring to the following example which is given to illustrate the invention rather than to limit its scope.

### EXAMPLE I

Methods and Materials

Plasmids

pMPG-EGFR-Ecoil

The EGFR signal sequence was amplified by PCR with the primers 5′-ATMGMTGC GGCCGCATGC GACCCTCCGG GACG-3′ (SEQ ID NO:3) and 5′-GGACTAGTCT TTTCCTCCAG AGCCCG-3′ (SEQ ID NO:4), which allowed the insertion of a Notl site at the 5′ terminus and Spel site at the 3′ terminus. pcDNA3-ErB1 (Lenferink et al., J. Biol. Chem., 275(35), 26748-26753, 2000) was used as template. 6 His and E-coil sequence were amplified by PCR using the primers 5′-CTAGCTAGCC ATCACCACCA TCATCAC-3′ (SEQ ID NO:5) and 5′-CCGCTCGAGT GATCCTCCAC C-3′ (SEQ ID NO:6) with the insertion of Nhel site at the 5′ terminus and Xhol site at the 3′ terminus. pcDNA3-E5coil was used as template (De Crescenzo G, et al., J Mol Biol. 328(5): 1173-1183, 2003). The transmembrane and cytoplasmic parts of EGFR were amplified with the primers 5′-CCGCTCGAGC CGTCCATCGC CACTGGG-3′ (SEQ ID NO:7) and 5′-CGGATATCTC ATGCTCCMT AAATTC-3′ (SEQ ID NO:8) with the insertion of Xhol site at the 5′ terminus and EcoRV site at the 3′ terminus. pcDNA3-ErB1 was used as template. The three fragments were cut with appropriate enzymes and ligated, then inserted into Notl and EcoRV sites of the vector pMPG, which express both BFP and hygromycin-resistant genes from independent cassettes.

CMV-FBK3/K4/K5

The oligonucleotide 5′-GGATCTGGAT CAGGTTCAGG AGTGGATCC-3′ (SEQ ID NO:9) containing a linker of 5 gly-ser and BamHl site was inserted at C terminus of the fiber gene under the control of CMV5 promoter in pCMV-FB-BFP plasmid. K-coil sequences were amplified with the primers 5′-CGCGGATCCA AGGTATCCGC TTTAAAG-3′ (SEQ ID NO:10) and 5′ CGCGGATCCC AATTGTTACT CCTTCAGAGC ACT-3′ (for K3: SEQ ID NO:11), or 5′-CGGGATCCCA ATTGTTATTC CTTCMGGCT GACAC-3′ (for K4: SEQ ID NO:12), or 5′-CGGGATCCCA ATTGTTACTC TTTAMGTGCT GA-3′ (for K5: SEQ ID NO:13), using pcDNA3-K5coil (also referred to sometimes as pcDNA3-HaKR1) (De Crescenzo et al., J Biol Chem, 279(25): 26013-26018, 2004) as template, digested by BamHI then inserted in the BamHI site of previously constructed plasmid pCMV-FB-BFP. A Munl site was incorporated in the amplified K-coil sequences after the stop codon.

CMV-FK4m and pE4-FK4m

A quikchange site-directed mutagenesis kit (Stratagene) was used for the mutation of fiber at aa 408. CMV-FK4 was amplified using the primers 5′-ACCACACCAG CTCCAGAGCC TMCTGTAGA CTAAATGC-3′ (SEQ ID NO:14) and 5′-GCATTTAGTC TACAGTTAGG CTCTGGAGCT GGTGTGGT-3′ (SEQ ID NO:15), which contain the mutation. The PCR condition is 1 cycle of 30 seconds at 95C.° and 16 cycles of 30 seconds at 95C,°, 1 minute at 55C° and 25 minutes at 68C.°. The methylated and no-mutated parental DNA template was then digested by Dpnl, while the mutated neo-synthesized plasmids are unmethylated, therefore uncleaved by Dpnl. They were then amplified in DH5α bacterial cells after transformation. For pE4-FBK4m, the plasmid CMV-FBK4m was then cut by Mun1 and Nhe1, digested fragment containing the modified part of fiber gene was inserted into MunI and Nhel-digested pE4 plasmid, which contains Ad sequence (84,5 mu to 100 mu) including fiber gene. The modified part of the fiber replaced the wildtype (wt) fiber in pE4 plasmid.

pE4-FK4mmRGD

The plasmid pE4-FK4m is mutated at aa409 by quickchange site-directed mutagenesis kit as described for CMV-FK4m. The two primers used for this mutation are 5′-ACCACACCAG CTCCAGAGGC TMCTGTAGA CTAAATGC-3′ (SEQ ID NO:16) and 5′-GCATTTAGTC TACAGTTAGC CTCTGGAGICT GGTGTGGT-3′ (SEQ ID NO:17). This plasmid pE4-FK4mm is then used to create pE4-FK4mmRGD. A fragment containing RGD sequence at HI-loop of Fiber is constructed by 2 steps PCR: at first two fragments FA and FB were amplified using primers 5′-CCGGTCCTCC MCTGTG-3′ (SEQ ID NO:18) with 5′- CAGTCTCCGC GGCAGTCACA ACCTCCTGTT TCCTGTGTAC CG-3′ (SEQ ID NO:19) and 5′-TGTGACTGCC GCGGAGACTG TTTCTGCGGA GGTGACACM CTCCMGTGC A-3′ (SEQ ID NO:20) with 5′-GGCCMTTGT TATTATTCCT TCMGGCTGA CAC-3′ (SEQ ID NO:21). pE4-FK4mm was used as template. Then FA and FB, which contain overlapping sequences between them, were mixed together and amplified by PCR; the resulting fragment FC is composed by both FA and FB. FC was then digested by Nhel and Munl, and inserted into pE4-FK4mm cut by the same enzymes. Fragment FK4mm-RGD were also amplified by PCR, and inserted into plasmid pAdCMV5.

Cells

293, HeLa and A549 cells were maintained in Dulbecco's Modified Eagle's Medium (DMEM) (Gibco), supplemented With 10% heat-inactivated calf serum. HeLa-rtTA and A549-tTA have been described previously (Massie B, et al., J Virol, 72(3): 2289-2296,1998).

Stable cell line 293E cells was generated by transfection of 293 cells with pMPG-EGFR-Ecoil/BFPq (5 μg). This transfection was done using the polyethylenimine (PEI) (7.5 μg) precipitation method. 48 h post-transfection, the cells were subjected to selection for 3 weeks with hygromycin (400μg/ml). The cells expressing the highest level of BFP reporter protein were distributed into 96-well plates and expanded under the selective pressure with hygromycin.

Viruses

Ad/GFP Viral Construction

AdEasy™ deleted in E1 and E3 regions (QBiogene) was used to produce Ad/GFP by homologous recombination with a transfer vector containing GFP gene under TR5/CuO promoter (Mullick, A., Konishi Y., Lau P., and Massie, B. A cumate-inducible system for regulated expression in mammalian cells (International publication WO02/088346). 100 ng of AdEasy and 1 μg of Pmel-linearized transfer vector were used for transformation of BJ5183 bacterial cells by electroporation (2.5 KV). The resultant Ad/GFP contains the reporter GFP gene in the E1 region.

AdFK4m Viral Construction

AdEasy deleted in E1 and E3 regions was cut with Munl, Pacl and Spel, and the digested fragments Munl/Pacl and Pacl/Spel were ligated with another fragment Munl/Spel derived from plasmid pE4-FK4m. This later plasmid contains Ad sequence (84.5 mu to 100 mu) including fiber gene, which has a mutation at aa 408 (S->E), and a K-coil sequence inserted at C-terminus.

AdFK4m/GFP Viral Construction

AdFK4m was used to produce AdFK4m/GFP by homologous recombination with a transfer vector containing GFP gene under TR5/CuO promoter 100 ng of AdFK4m and 1 ug of Pmel-linearized transfer vector were used for transformation of BJ5183 bacterial cells as described for Ad/GFP.

AdFK4mmRGD/GFP Viral Construction

pE4-FK4mmRGD. were cut by Kpnl and Pacl. 200 ng of digested fragments containing Fiber-RGD were cotransfected with 100 ng of AdEasyΔfibre/GFP digested by Srfl into BJ5183, the recombinant AdFK4mmRGD/GFP was then selected.

Rescue of Ad Viruses

5 μg of viral DNA Ad/GFP and AdFK4m/GFP were cleaved with Pacl, then respectively transfected into 293E or 293 cells by PEI precipitation method. Cells were harvested after 21 days when they showed cytopathic effect. After three cycles of freeze-thaw to release Ad particles, 293E or 293 cells were infected with half of the cell lysate to propagate the viruses. The infectious titers were determined by measuring the GFP expression in 293E cells using flow cytometry (λ=525nm).

Proteins Expressing Analysis

5 μg of DNA was used for cell transfection by PEI precipitation method. 48 h later, cells were collected, washed with phosphate buffered saline (PBS), lysed in 62.5 mM Tris-HCl (pH6.8), 10% glycerol, 2%SDS, 2.5% 2-mercaptoethanol (denaturing condition) or non-reducing buffer (the same buffer except 1%SDS without 2-mercaptoethanol, followed by sonication. 25-50 μg of total protein extract was loaded onto acrylamide gel. After transfer, the nitrocellulose membrane was blocked with PBS containing 5% dry milk, 0.1% tween 20™ during 1 h at room temperature, and then probed with a monoclonal antibody against EGFR (1:1000), fiber protein (1:500) (Neomarkers) or histidine (1:500) (Qiagen) overnight at 4° C. Proteins were then detected by using anti mouse peroxidase (1:5000) and the ECL™ chemiluminescence kit (Amersham).

Cytofluorometry

Cells were dislodged from tissue culture plate by cell dissociation solution (sigma), centrifuged at 1000 rpm, and resuspended at 1×10⁶ cells/ml in complete medium containing 10% serum, then incubated with 10 μl antibody against to His, followed by incubation of 6 μl of Alexa green fluor 488 coat anti-mouse IgG (Molecular probes A-1100). Each incubation step was done during 1 h on ice. The cells were analyzed by FACScan™ cytometer at λ of 525nm.

Virus Transduction Assays

5×10⁵ Cells were seeded on 12-well plates and incubated with virus during 2 days at 37° C. Prior to infection, cells were incubated with or without K-coil peptide (2 μg) or soluble fiber protein (2 μg) in 200 μl DMEM medium for 1 h at 37° C. Peptides remained present during virus infection when 300p1 of virus were added. Transduction efficiency was evaluated by monitoring GFP expression by flow cytometry analysis.

### RESULTS

Generation of a 293 Cell line (293E) Expressing a Pseudoreceptor (EGFR-Ecoil)

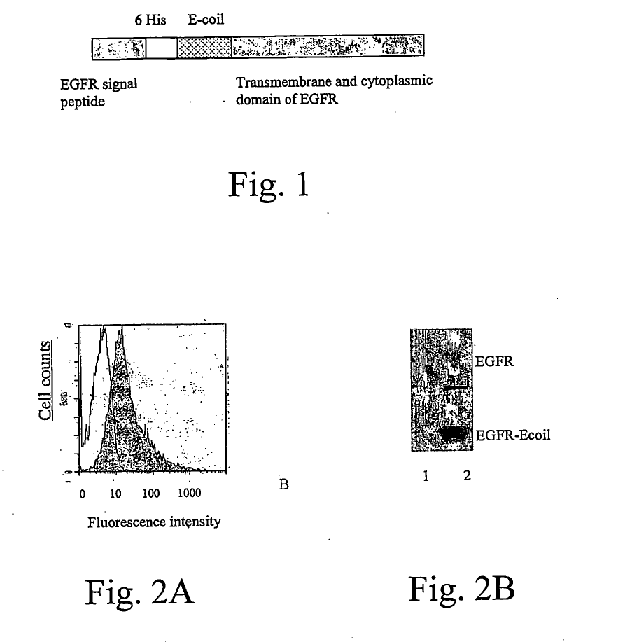

An artificial peptide E-coil (5 heptads of EVSALEK) was chosen for the construction of EGFR-Ecoil pseudoreceptor. Using such an artificial ligand should exclude the possibility of accidental in vivo binding of a modified AdV to receptors other than the selected target. Gene encoding the fusion protein EGFR-Ecoil (FIG. 1) was cloned in a mammalian expression vector pMPG under the control of a modified CMV promoter. EGFR-Ecoil is composed of the signal sequence of EGFR, 6 His, E-coil sequence, the transmembrane and cytoplasmic parts of EGFR. The EGFR signal sequence directs Ecoil to the cell surface and the EGFR transmembrane domain anchors the receptor in the plasma membrane. The 6 His permit detection of the protein by immunoblot and flow cytometry analysis. The resultant plasmid contains also the gene for hygromycin selection and BFP (blue fluorescent protein) reporter expressed from independent cassettes.

Stable cell lines (293E) were generated by transfection of 293 cells with the plasmid pMPG-EGFR-Ecoil, followed by selection in the presence of hygromycin. The BFP positive cells were sorted using the multiwell automated cell deposition system and clonal distribution was visually checked.

Five of the best clones as assessed by BFP expression were further characterized for EGFR-Ecoil pseudoreceptor surface expression by flow cytometry of cells following incubation with the anti-His Ab. FIG. 2A shows the profile of the best clone displaying a marked increase in cell fluorescence (293E cells) as compared to 293 cells (without pMPG-EGFR-Ecoil). The median fluorescence for 293E cells was 732, versus 11 for 293 cells. Most of the other dories had a similar profile. This result demonstrated that the pseudoreceptors EGFR-Ecoil were displayed on the cell surface. In FIG. 2A, cells were detached by cell dissociation solution (sigma), resuspended at 1×10⁶ cells/ml, and incubated with 10 ul of anti-his Ab, followed by the incubation of 6ul of anti-mouse Ab*. The fluorescence intensity is plotted on a logarithm scale on the x-axis. The empty peak represent 293 cells while the shadowed peak represent 293E cells.

The EGFR-Ecoil expression in two clones was confirmed by Western blotting using anti-EGFR-Ecoil antibody (anti-erb1), which can recognize the cytoplasmic part of EGFR. As shown in FIG. 2B, the expression of the pseudoreceptor is much stronger than the endogenous EGFR in 293E cells (line 2), while this pseudoreceptor is not detected in 293 parental cells (line 1). In FIG. 2B, 293 cells (1) and 293E cells (2) lysates were subjected to SDS-PAGE (10%), transferred to nitrocellulose, and probed with an anti-erb-1 antibody at dilution of 1:1000. The bands corresponding to EGFR and EGFR-Ecoil are identified.

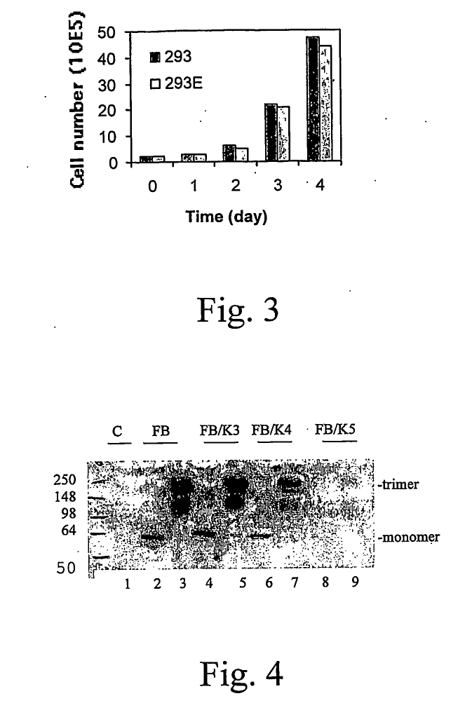

The growth rate of the selected 293E cells was compared with. parental cells (FIG. 3). Both cells showed similar profile, which indicate that the expression of EGFR-Ecoil pseudoreceptor did not significantly affected the cell physiology. In FIG. 3, cells were seeded at 2×10⁵ in DMEM medium supplemented with 10% of heat-inactivated fetal bovine serum, and counted on a daily basis until the monolayers reach confluency.

Construction of AdV Containing Chimeric Fiber Incorporating Kcoil in the knob (AdF4Km/GFP)

The artificial heptad K-coil (KVSALKE), which has high affinity to E-coil, was selected as the ligand to be inserted into the fiber knob of AdV. A crucial requirement for successful fiber modification by incorporation of a peptide is that this should neither change its conformation nor its normal function. Two questions have been therefore addressed: Is the 5 heptads (35 aa) segment of K-coil small enough to be incorporated into fiber without changing its trimerization, which is essential for fiber incorporation into the capsid and proper virus assembly? If the size of the K-coil motif was varied by eliminating 1 or 2 heptad sequences, will it then retain an affinity to E-coil peptide high enough to insure efficient binding of the AdV to the pseudoreceptor?

Different repeats of E-coil and K-coil have been synthesized and their interaction has been analyzed by BIACORE (De Crescenzo G, et al., Biochemistry, 42(6): 1754-1763, 2003). E5 (E-coil of 5 repeats) binds K5 (K-coil of 5 repeats) with very high affinity (Kd =63pM). The association capacity decreased with the number of the heptad: Kds are 14nM and 7 μM respectively for E5/K4 and E5/K3 interaction. Clearly, reducing the number of heptad by 2 in K-coil motif dramatically decreased it's binding to E-coil.

In the present invention, the suitable number of K-coil heptad was also investigated for their incorporation in fiber without disturbing the fiber trimerization. Chimeric fiber genes with 3, 4 or 5 heptads of K-coil motif (K3, K4 or K5) at the C-terminus were cloned respectively into the vector pAdCMV5K7BFP under the CMV5 promoter. In order to optimize the accessibility of the K-coil in fiber to the EGFR-Ecoil pseudoreceptor, a flexible linker made of 5 glycine residues was added between the fiber and K-coil. The recombinant proteins were analyzed by western blotting under denaturing (FIG. 4, lanes 1, 2, 4, 6, and 8) and non-denaturing conditions (lanes 3, 5, 7 and 9). In non-denaturing condition, the trimer forms of FB/K3 (lane 5) and FB/K4 (lane 7) are at same level as wt fiber (lane 3). In contrast, the overall expression of FB/K5 (lane 9) was dramatically decreased while its trimerization was slightly affected. Note that the anti-fiber antibody used in this western blot preferentially recognized the trimeric fiber. This result shows that, both 3 and 4 heptads of K-coil incorporated in fiber did not compromise the expression nor the trimerization of these proteins. In FIG. 4, 293 cells were transiently transfected with pAd-CMV-GFP control plasmid (1), or plasmids expressing wt fiber (2 and 3), fiber/K3 (4 and 5), fiber/K4 (6 and 7) and fiber/K5 (8 and 9). Cells lysates in either denaturing (1, 2, 4, 6, and 8) or non-denaturing conditions (3, 5, 7 and 9) were run on SDS-PAGE (10%). The proteins were transferred to nitrocellulose, and detected by an anti-fiber antibody (1:500). At the left are shown the positions of molecular weight standards in kilodaltons.

Given its higher affinity for E-coil, K4 was selected as the ideal candidate to be inserted into virus capsid. An AdV was then constructed in which the fiber gene contained K4 at C terminus in addition to a mutation (S ->E) at aa 408 known to abolish the fiber interaction with CAR. This recombinant virus has also a reporter gene encoding for GFP in the E1 region (AdK4m/GFP). The viral DNA generated in E coi by homologuous recombination was transfected in 293E cells to produce the virus. A control virus Ad/GFP that contains wt fiber and GFP under the same promoter was also constructed.

Transduction of 293E Cells by AdF4Km/GFP

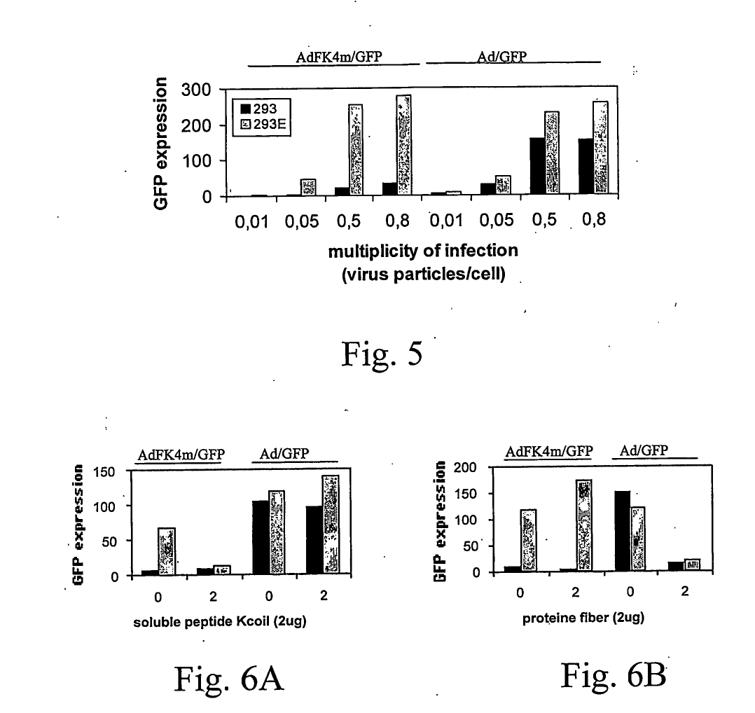

In order to test whether the membrane-anchored EGFR-Ecoil could serve as an artificial receptor for AdFK4m/GFP, both 293 and 293E cells were infected with this virus at MOI of 0.01; 0.05; 0.5, or 0.8. Although GFP expression is controlled by the tetracycline-regulated promoter in the expression cassette, due to leaky expression of the promoter and massive gene amplification following replication, GFP expression was easily detectable in 293 cells without the tetracycline trans-activator (tTA) as previously shown (Massie B, et al., J Virol, 72(3): 2289-2296, 1998). Transduction efficiencies were evaluated two days later by measuring GFP expression in infected cells using flow cytometry analysis. As shown in FIG. 5, 293 cells without the pseudoreceptor are barely transduced by AdFK4m/GFP, in contrast to the wt Ad/GFP, especially at low MOls. In FIG. 5, cells were infected with equal amounts of virus particles as indicated, 48 h later, GFP expression in infected cells was analyzed by flow cytometry. This result is consistent with the expected reduced transduction efficiency of AdFK4m/GFP with the fiber mutation at aa 408. In sharp contrast, the transduction efficiencies in 293E cells of AdFK4m/GFP, as compared with 293 cells, were increased 7-fold at MOI of 0.01, 24-fold at MOI 0.05, 11-fold at MOI 0.5 and 8-fold at MOI of 0,8, and they reach the same transduction level as wt virus Ad/GFP. As expected, the control Ad/GFP infects 293E cells at the same level as 293 cells._These results indicated that AdFK4m/GFP infects 293E cells via a CAR-independent cell entry pathway. The expression of trimer form of modified-fiber in infected 293E cells was confirmed by immunoblot.

Competitive inhibition assays were performed in order to confirm that 293E transduction by AdF4Km/GFP required the specific binding of the Ad vector to the pseudoreceptor via E-coil/K-coil interaction. In FIG. 6A and 6B, 293 (Black bar) and 293E (Grey bar) cells were infected with AdFK4m/GFP or Ad/GFP at a MOI of 0.05. Prior to addition of virus, cells were incubated for 1 h with 0 or 2 μg of K-coil soluble peptide (6A) or soluble Ad5 fiber (6B). GFP expression was monitored by flow cytometry analysis at 48 h pi. When cells were pre-incubated with K-coil, AdFK4m/GFP mediated GFP gene transfer in 293E cells was inhibited by 80% while no effect was observed for the transduction of 293 parental cells. For the control virus Ad/GFP meditated-transduction, no inhibition was observed in either 293 or 293E cells (FIG. 6A). By contrast, fiber inhibits the Ad/GFP transduction in both 293 and 293E cells, while it has not any effect for AdFK4m/GFP mediated-transduction in 293E cells (FIG. 6B). These results clearly demonstrated that binding of AdFK4m/GFP to the pseudoreceptor via E-coil/K-coil interaction mediates virus infection to 293E cells in the absence of fiber-CAR interaction.

In conclusion, the complementary components consisting of modified Ad virion and cell line together constitute a novel system that permits the fiber receptor-independent propagation of tropism-modified AdVs.

Characterization of Virus Growth Kinetics

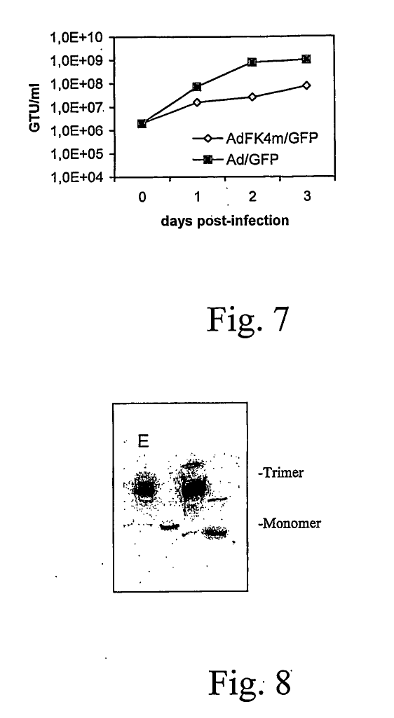

Virus growth rate of the modified virus AdFK4m/GFP were tested in comparison with Ad/GFP (FIG. 7). 293E cells were infected at an MOI of 2 active virus particles/cell with both virus, and the titers were determined by measuring the GFP expression at 1, 2, and 3 days post-infection. In FIG. 7, on day 0, 293E cells were infected with Ad/GFP or AdFk4m/GFP at MOI of 2. At days 1, 2, or 3 post-infection, the cells were harvested and freeze-thawed, the infectious particles titers, which were expressed as GTU (GFP Transfer Unit) 1 ml of cell lysate, were determined measuring the GFP expression flow cytometry analysis. The growth curves for both virus showed similar shapes and no lag was observed in recombinant virus growth. However, the production of infectious modified virus was inferior to the virus with wt fiber. This could be due to suboptimal level of expression of the EGFR-Ecoil pseudoreceptbr in 293E cells or to suboptimal expression of K4-fiber in AdFK4m/GFP.

Gene Delivery by Genetically Modified-Ad Vector

Having generated a CAR-ablated AdV with a K-coil modified fiber and 293E cells for its amplification, a tropism-modified virus was then constructed. As an example, RGD motif was inserted into the HI-Loop of fiber in order to target virus to cellular proteins integrina. At same time, the aa409 of fiber-RGD was also modified (P->A) to further ablate fiber's interaction with it's natural receptor CAR. The trimerization of this modified-fiber was tested by immunoblot after transient transfection of 293 cells (FIG. 8). In FIG. 8, plasmids allowing the expression of wt fiber (1 and 2), RGD (3 and 4)-containing fiber were transfected into 293 cells, 48 h later, the trimer form of fiber-expression (1 and 3) was detected as described in FIG. 4. The FK4mm-RGD modified fiber (lane 3) showed same trimer expression level as the wild-type fiber (lane 1). The AdK4mmRGD/GFP virus was then produced by transfection of 293E cells.

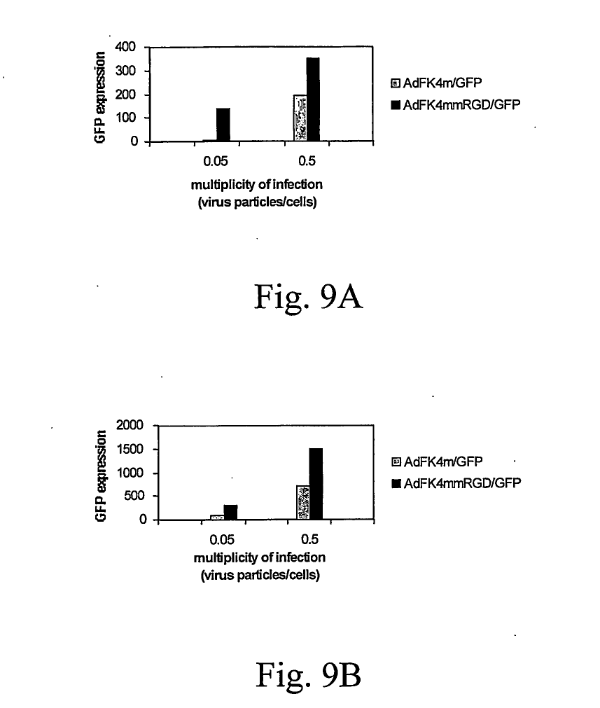

The effect of RGD incorporation in the modified fiber (FK4mm-RGD) was first tested by measuring the gene delivery efficiency of AdFK4mmRGD/GFP in E1-complementing cells. AdFK4mmRGD/GFP was incubated at different MOls with 293 (FIG. 9A) and 293E (FIG. 9B) cells, and GFP expression was analyzed by flow cytometry 2 or 3 days later as a measure of transduction efficiency. As compared with the CAR-ablated virus AdFK4m/GFP, AdFK4mmRGD/GFP showed, in 293 cells, a significant increase in GFP expression, especially at lower MOI, indicating that gene delivery was improved by the addition of RGD in the modified fiber. In Ecoil-containing 293E cells, AdFK4mmRGD/GFP transduced 2 to 3 times better than AdFK4m/GFP at both MOls used. As expected, the transduction level in 293E cells is higher than 293 cells, since the virus can also enter into cells via the pseudoreceptor EGFR-Ecoil.

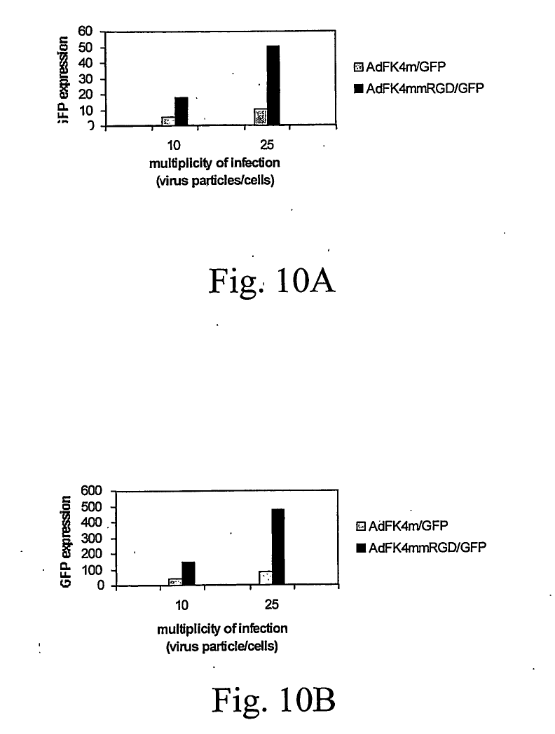

AdFK4mmRGD/GFP's transduction efficiency was also tested in cells that do not support virus replication (HeLa and A549) (FIGS. 10A and 10B). In FIGS. 10A and 10B, 5×10⁵ HeLa-rtTA (10A) and A549-tTA cells (10B) were infected, with equal amounts of virus particles AdFK4mmRGD/GFP and AdFK4m/GFP at indicated. MOls. The resulting GFP expression (y axis) was analyzed by flow cytometry 2 days later. Detection of GFP expression in such cells was facilitated by the expression of the tetracycline-inducible transactivators (tTA or rtTA). In both cell lines the transduction efficiency of AdFK4mmRGD/GFP was increased by a factor 3 to 6 fold as compared to AdFK4m/GFP depending on the MOI (FIGS. 10A and 10B). Taken together, these results demonstrated that the modified vector AdFK4mmRGD/GFP restore the cell transduction via the RGD motif-added in fiber.

In conclusion, the complementary components comprising modified Ad virion and cell line together constitute a novel system that permits the fiber receptor-independent propagation of tropism-modified AdVs. One of the main advantages of this system is the possibility of re-targeting, either through direct incorporation of ligands in the capsid, or through the construction of adapters (coil-fused ligands).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A modified virus ablated of its natural receptors interactions with an unmodified or non-naturally occurring cell, said modified virus comprising a non-native polypeptide, said modified virus having an altered tropism conferred by said non-native peptide, and replicating only in cells that can interact with said non-native peptide, said virus being incapable of infecting a cell through a CAR-dependent entry pathway.

2. The modified virus of claim 1, which is derived from a virus selected from the group consisting of adenovirus, retrovirus, lentivirus, adeno-associated virus, Reoviridae, Picornaviridae, Parvoviridae, Papovaviridae and Caliciviridae.

3. The modified virus of claim 1, which is derived from human adenovirus.

4. The modified virus of claim 1, which is derived from human adenovirus serotype 2 or 5.

5. The modified virus of claim 4, wherein said non-native polypeptide replaces, is incorporated into, or forms a fusion protein with, a viral protein component of the wild type virus.

6. The modified virus of claim 5, wherein said viral protein component is an adenoviral fiber protein.

7. The modified virus of claim 6, wherein said non-native polypeptide is incorporated into an adenoviral fiber protein such that the wild-type fiber knob or cell binding domain thereof is removed.

8. The modified virus of claim 1, wherein said non-native polypeptide is or comprises a combinatorial protein or an affibody.

9. The modified virus of claim 1, wherein said non-native polypeptide comprises one or more sequence from a bacterial receptor ligand.

10. The modified virus of claim 1, wherein said non-native polypeptide comprises at least one repeat of a sequence as set forth in SEQ ID NO:1.

11. The modified virus of claim 1, wherein said non-native polypeptide comprises at least one repeat of a sequence as set forth in SEQ ID NO:2.

12. The modified virus of claim 1, wherein said non-native polypeptide binds a non-naturally occurring production cell or permissive cell.

13. The modified virus of claim 1, further comprising a retargeting adapter comprising: i) a binding moiety for binding the non-native polypeptide and ii) a further binding moiety of a receptor for retargeting said virus on cells expressing said receptor.

14. The modified virus of claim 13, wherein said non-native polypeptide comprises at least one repeat of a sequence as set forth in SEQ ID NO:1 and said binding moiety for binding the non-native polypeptide comprises at least one repeat of SEQ ID NO:2.

15. The modified virus of claim 13, wherein said non-native polypeptide comprises at least one repeat of a sequence as set forth in SEQ ID NO:2 and said binding moiety for binding the non-native polypeptide comprises at least one repeat of SEQ ID NO:1.

16. The modified virus of claim 13, wherein said adapter binds to the non-native polypeptide through non-covalent physical forces selected from the group consisting of van der waals forces, electrostatic forces, stacking interactions, hydrogen bonding and steric fit.

17. The modified virus of claim 1, wherein said non-native polypeptide comprises a cleavage site positioned in a location that enables a further binding moiety of a receptor to be added on the modified virus for retargeting said virus on cells expressing said receptor.

18. The modified virus of claim 17, wherein the binding moiety is capable of binding to a cell specific ligand.

19. The modified virus of claim 1, which further comprises a site for insertion of one or more desired therapeutic genes or nucleic acid molecules.

20. A cell containing a modified virus as defined in claim 1.

21. A permissive cell for a modified virus as defined in claim 1, which is capable of being cultured to propagate said modified virus.

22. A non-naturally occurring permissive cell expressing a surface receptor recognizing or binding a non-native polypeptide as defined in 1.

23. A non-naturally occurring permissive cell expressing a surface receptor recognizing or binding a non-native polypeptide as defined in claim 10, wherein said surface receptor comprises at least one copy of the amino acid sequence as set forth in SEQ ID NO:2.

24. A non-naturally occurring permissive cell expressing a surface receptor recognizing or binding a non-native polypeptide as defined in claim 11, wherein said surface receptor comprises at least one copy of the amino acid sequence as set forth in SEQ ID NO:1.

25. A method for producing a modified virus as defined in claim 1 in cell culture, comprising the steps of: i) genetically modifying a virus to produce a modified virus ablated of its natural receptors interactions with an unmodified or non-naturally occurring cell, said modified virus comprising a non-native polypeptide, said modified virus having an altered tropism conferred by said non-native peptide, and replicating only in cells that can interact with said non-native peptide; ii) infecting permissive cells with said modified virus; and iii) culturing said cells to produce the virus.

26. The method of claim 25, further comprising a step of iv) harvesting the modified virus produced.

27. The method of claim 26, further comprising a step of v) purifying the modified virus produced.

28. A pharmaceutical composition comprising a modified virus as defined in claim 1 and a pharmaceutically acceptable carrier or excipient.

29. A reagent kit comprising: i) a modified virus as defined in claim 1 ii) a permissive cell expressing a surface receptor recognizing or binding a non-native polypeptide, said cell being capable of being infected by said modified virus.

29. A medicament or a precursor thereof comprising a virus as defined in claim 1.


SEQUENCE LISTING (21 sequences)

SEQ ID NO: 1 - protein, 7 aa, Artificial Sequence
  E-coil
EVSALEK

SEQ ID NO: 2 - protein, 7 aa, Artificial Sequence
  K-coil
KVSALKE

SEQ ID NO: 3 - DNA, 34 bp, Artificial Sequence
  Primer to amplify EGFR signal sequence
ataagaatgcggccgcatgcgaccctccgggacg

SEQ ID NO: 4 - DNA, 26 bp, Artificial Sequence
  Primer to amplify EGFR signal sequence
ggactagtcttttcctccagagcccg

SEQ ID NO: 5 - DNA, 27 bp, Artificial Sequence
  Primer to amplify 6 His and E-coil sequence
ctagctagccatcaccaccatcatcac

SEQ ID NO: 6 - DNA, 21 bp, Artificial Sequence
  Primer to amplify 6 His and E-coil sequence
ccgctcgagtgatcctccacc

SEQ ID NO: 7 - DNA, 27 bp, Artificial Sequence
  Primer to  amplify the transmembrane and cytoplasmic parts of EGFR
ccgctcgagccgtccatcgccactggg

SEQ ID NO: 8 - DNA, 26 bp, Artificial Sequence
  Primer to  amplify the transmembrane and cytoplasmic parts of EGFR
cggatatctcatgctccaataaattc

SEQ ID NO: 9 - DNA, 29 bp, Artificial Sequence
  oligonucleotide containing a linker of 5 gly-ser and BamHI site
ggatctggatcaggttcaggagtggatcc

SEQ ID NO: 10 - DNA, 27 bp, Artificial Sequence
  Primer to amplify K-coil sequence
cgcggatccaaggtatccgctttaaag

SEQ ID NO: 11 - DNA, 33 bp, Artificial Sequence
  Primer to amplify K-coil sequence K3
cgcggatcccaattgttactccttcagagcact

SEQ ID NO: 12 - DNA, 35 bp, Artificial Sequence
  Primer to amplify K-coil sequence K4
cgggatcccaattgttattccttcaaggctgacac

SEQ ID NO: 13 - DNA, 32 bp, Artificial Sequence
  Primer to amplify K-coil sequence K5
cgggatcccaattgttactctttaagtgctga

SEQ ID NO: 14 - DNA, 38 bp, Artificial Sequence
  Primer to amplify CMV-FK4
accacaccagctccagagcctaactgtagactaaatgc

SEQ ID NO: 15 - DNA, 38 bp, Artificial Sequence
  Primer to amplify CMV-FK4
gcatttagtctacagttaggctctggagctggtgtggt

SEQ ID NO: 16 - DNA, 38 bp, Artificial Sequence
  Primer to amplify mutated plasmid pE4-FK4m
accacaccagctccagaggctaactgtagactaaatgc

SEQ ID NO: 17 - DNA, 38 bp, Artificial Sequence
  Primer to amplify mutated plasmid pE4-FK4m
gcatttagtctacagttagcctctggagctggtgtggt

SEQ ID NO: 18 - DNA, 17 bp, Artificial Sequence
  Primer
ccggtcctccaactgtg

SEQ ID NO: 19 - DNA, 42 bp, Artificial Sequence
  Primer
cagtctccgcggcagtcacaacctcctgtttcctgtgtaccg

SEQ ID NO: 20 - DNA, 51 bp, Artificial Sequence
  Primer
tgtgactgccgcggagactgtttctgcggaggtgacacaactccaagtgca

SEQ ID NO: 21 - DNA, 33 bp, Artificial Sequence
  primer
ggccaattgttattattccttcaaggctgacac